(19) United States
(12) Patent Application Publication
(10) Pub. No.: US 2016/0209875 A1
(43) Pub. Date: Jul. 21, 2016

(54) WEARABLE DISPLAY DEVICE

(71) Applicant: SAMSUNG DISPLAY CO., LTD., Yongin-City (KR)

(72) Inventor: Young Chan KIM, Incheon (KR)

(21) Appl. No.: 14/800,036

(22) Filed: Jul. 15, 2015

(30) Foreign Application Priority Data
Jan. 15, 2015 (KR) 10-2015-0007459

(51) Int. Cl. G06F 1/16; A61B 5/00; A61B 5/01; G09G 3/32

(52) U.S. Cl. CPC G06F 1/1652; G06F 1/163; G09G 3/3208; A61B 5/4266; A61B 5/01

(57) ABSTRACT

A wearable display device including a stretchable display wearably providable on a human body to display an image; a body sensor on a rear of the stretchable display to contact the human body; and a modifier in the stretchable display, the modifier increasing an area of the stretchable display based on a sensing signal generated from the body sensor.

### CROSS-REFERENCE TO RELATED APPLICATION

Korean Patent Application No. 10-2015-0007459, filed on Jan. 15, 2015, in the Korean Intellectual Property Office, and entitled: “Wearable Display Device,” is incorporated by reference herein in its entirety.

### BACKGROUND

1. Field

Embodiments relate to a wearable display device.

2. Description of the Related Art

With the development of electronic technologies, the use of portable electronic devices, such as a smart phone, a tablet PC, a laptop PC, personal digital assistants (PDAs), has been increased.

Recently, to increase portability of the portable electronic devices, the importance of wearable devices that the portable electronic devices are put on a human body has been steadily increased, and therefore a study on applications thereof has been actively conducted.

The wearable device has a structure in which a display displaying an image may be put on, e.g., a wrist or a forearm. Therefore, the wearable device may be conveniently put on, and both of a user's hands may be used freely, and therefore various types of wearable products have been considered.

The above information disclosed in this Background section is only for enhancement of understanding of the background of the invention and therefore it may contain information that does not form the prior art that is already known in this country to a person of ordinary skill in the art.

### SUMMARY

Embodiments are directed to a wearable display device.

The embodiments may be realized by providing a wearable display device including a stretchable display wearably providable on a human body to display an image; a body sensor on a rear of the stretchable display to contact the human body; and a modifier in the stretchable display, the modifier increasing an area of the stretchable display based on a sensing signal generated from the body sensor.

The stretchable display may be stretchable and contractible in a length direction thereof in response to an external force, and the modifier may provide the external force to the stretchable display to increase a length of the stretchable display or restore the length of the stretchable display to an original state.

The body sensor may include a moisture sensor to sense sweat generated from the human body.

The body sensor may include a temperature sensor to sense body temperature.

The body sensor may include a moisture sensor to sense sweat generated from the human body, and a temperature sensor to sense body temperature.

The moisture sensor and the temperature sensor may be alternately disposed along a length direction of the stretchable display.

The modifier may include electromagnets inside a rear of the stretchable display, the electromagnets being spaced apart from each other at a predetermined interval along a length direction of the stretchable display.

The body sensor may sense a change in the human body and outputs the sensing signal to a driving controller of the stretchable display, and the driving controller may apply a current in a same direction to the electromagnet based on the sensing signal.

The electromagnet may form a same pole to generate a repulsive force and extend a length of the stretchable display by the generated repulsive force.

The stretchable display may include an organic light emitting diode.

The wearable display device may further include wearing parts provided at sides of the stretchable display to put the stretchable display on the human body.

The wearing part may include a band that is connected to sides of the stretchable display.

The band may include one portion that is connected to one end of the stretchable display and that has a rear provided with a hook protrusion, and another portion that is connected to another end of the stretchable display and that is provided with hook holes that are spaced apart from each other along a length direction of the band.

The wearing parts may be provided at edge portions along a length direction of the stretchable display.

The wearing part may include magnetic bodies installed at the edge portion of one end of the stretchable display; and metal bodies installed at the edge portion of another end of the stretchable display to be spaced apart from each other at a predetermined interval.

The wearing part may include hook protrusions installed at the edge portions of one end of the stretchable display; and hook holes installed at the edge portions of another end of the stretchable display to be spaced apart from each other at a predetermined interval.

Opposing ends of the wearing parts may overlap at an overlapped portion, the stretchable display may display an image in a remaining area other than an overlapped portion, and the driving controller may not apply a current to the electromagnet that is positioned at the overlapped portion of the wearing parts.

### BRIEF DESCRIPTION OF THE DRAWINGS

Features will be apparent to those of skill in the art by describing in detail exemplary embodiments with reference to the attached drawings in which:

FIG. 1 illustrates a perspective view of a wearable display device according to an embodiment.

FIG. 2 illustrates a front configuration diagram of the wearable display device according to the embodiment.

FIG. 3 illustrates a rear configuration diagram of the wearable display device according to the embodiment.

FIG. 4 illustrates a side configuration diagram of the wearable display device according to the embodiment.

FIGS. 5A to 5C illustrate diagrams showing an operation of a wearable display device according to the embodiment.

FIG. 6 illustrates a perspective view of a wearable display device according to another embodiment.

FIG. 7 illustrates a front configuration diagram of the wearable display device according to the other embodiment.

FIG. 8 illustrates a rear configuration diagram of the wearable display device according to the other embodiment.

FIG. 9 illustrates a side configuration diagram of the wearable display device according to the other embodiment.

FIG. 10 illustrates a perspective view of a wearable display device according to still another embodiment.

FIG. 11 illustrates a front configuration diagram of the wearable display device according to the other embodiment.

FIG. 12 illustrates a rear configuration diagram of the wearable display device according to the other embodiment.

FIG. 13 illustrates a side configuration diagram of the wearable display device according to the other embodiment.

### DETAILED DESCRIPTION

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing figures, the dimensions of layers and regions may be exaggerated for clarity of illustration. Like reference numerals refer to like elements throughout.

Further, in exemplary embodiments, since like reference numerals designate like elements having the same configuration, an exemplary embodiment is representatively described, and in other exemplary embodiments, only a configuration different from the exemplary embodiment will be described.

Throughout the present specification, unless explicitly described to the contrary, “including” or “comprising” any components will be understood to imply the inclusion of other elements rather than the exclusion of any other elements.

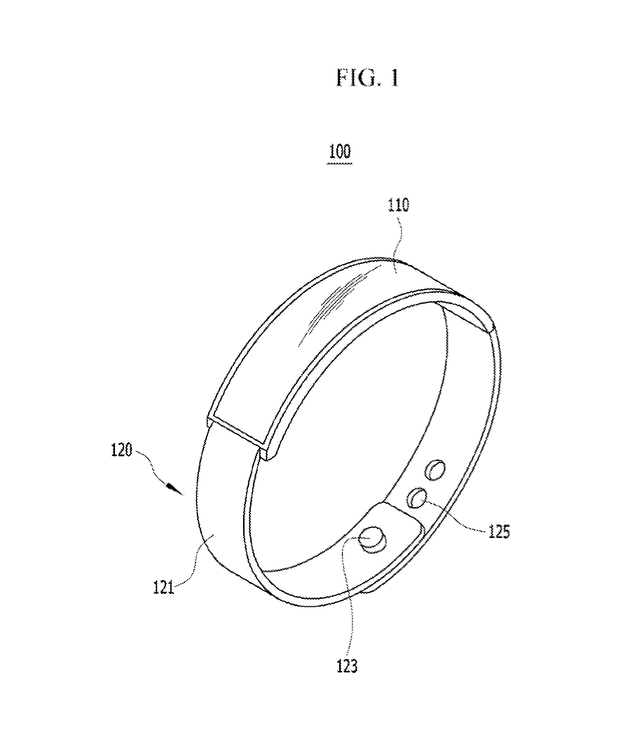

FIG. 1 illustrates a perspective view of a wearable display device according to an embodiment.

Referring to FIG. 1, a wearable device 100 according to an embodiment may be configured to be put on a user's body. For example, the wearable display device 100 may be put on a user's wrist or a forearm to display an image to a user.

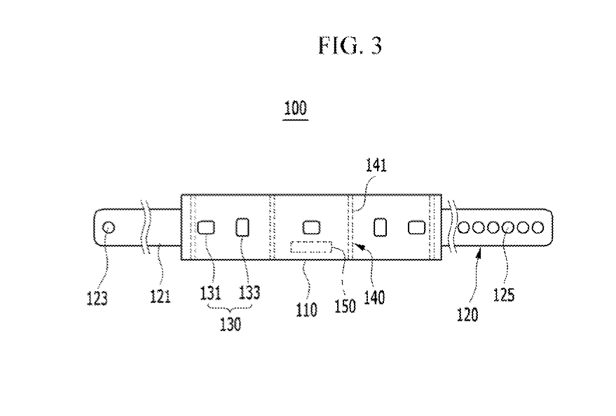

The wearable display device 100 may include, e.g., a stretchable display 110, a wearing part 120, a body sensor 130 (see FIG. 3), and a modifier 140.

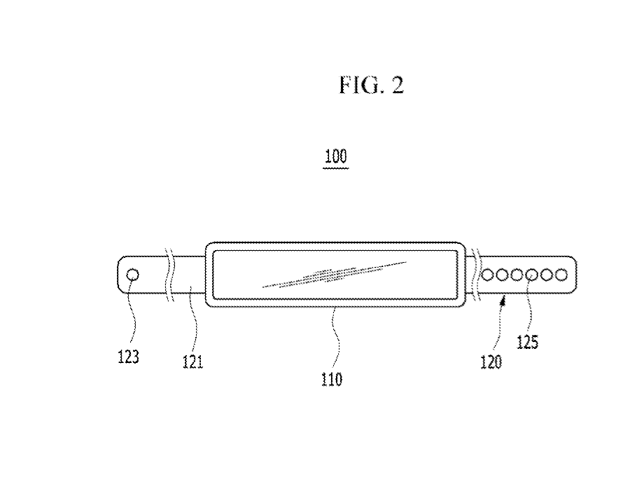

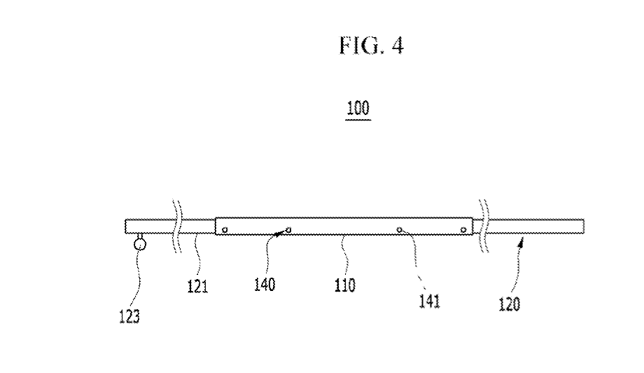

FIG. 2 illustrates a front configuration diagram of the wearable display device according to the embodiment. FIG. 3 illustrates a rear configuration diagram of the wearable display device according to the embodiment. FIG. 4 illustrates a side configuration diagram of the wearable display device according to the embodiment.

Referring to FIGS. 2 to 4, the stretchable display 110 may display an image. The stretchable display 110 may be made of a stretchable material that may be stretched by, e.g., an external pressure. The stretchable display 110 may have a form in which a whole area of the display may extend. For example, the stretchable display 110 may have an elastic restoring force (e.g., may be stretchable and contractible) and may be stretched and modified in various forms. The stretchable display 110 may have a plurality of pixels displaying an image. Here, the pixel may mean a minimum unit displaying an image. The stretchable display 110 may include, e.g., an organic light emitting diode (OLED) as a pixel displaying an image. Further, the stretchable display 110 may further include, e.g., at least one scan wiring, at least one data wire, a plurality of thin film transistors, and at least one capacitor which make the organic light emitting diode emit light. The stretchable display 100 may be formed to have various suitable structures. In an implementation, the stretchable display 100 may be a quadrangle in two dimensions.

In an implementation, the stretchable display 110 may have a quadrangular shape of which a length is longer than a width. The stretchable display 110 may be stretched and modified along a length direction, and may have an increased area, by or in response to an external force. For example, the stretched (e.g., stretching and/or contracting) direction of the stretchable display 110 may be a length direction.

In an implementation, the stretchable display 110 may be an organic light emitting display device including an organic light emitting diode. In an implementation, the stretchable display 100 may be, e.g., a liquid crystal display (LCD), a plasma display (PD), a field emission display (FED), an electrophoretic displays (EPD), an electrowetting display (EWD), or the like.

In an implementation, the stretchable display 110 according to an embodiment may be, e.g., stretchable, flexible, foldable, bendable, and/or rollable.

The wearing part 120 may be used to put and/or hold the stretchable display 110 on, e.g., the user's wrist or forearm. If the wearing part 120 is put on the user's wrist or forearm in the state of supporting the stretchable display 110, the wearing part 120 may be formed in a suitable shape or form. The wearing part 120 may have a suitable form, e.g., a watch form, a bracelet form, etc.

In an implementation, the wearing part 120 may be provided at sides, e.g., both sides, of the stretchable display 110. Herein, both sides mean both end portions of the stretchable display 110 based on the length direction of the stretchable display 110.

The wearing part 120 may include bands 121 connected to sides, e.g., both sides, of the stretchable display 110. The band 121 may be formed as, e.g., a rubber strip, and may have flexibility. The wearing parts 120 may be connected to ends, e.g., both ends, of the stretchable display 110 along or in the length direction of the stretchable display 110.

The band 121, e.g., one portion of the band 121, may be connected to one end of the stretchable display 110. A rear or distal end of the band 121 connected to the one end of the stretchable display 110 may be provided with a hook protrusion 123. In an implementation, the hook protrusion 123 may protrude on the rear or inner side of the band 121 to be integrally formed with the band or may be fixedly installed to the rear of the band, e.g., separately from the band 121.

The band 121, e.g., another portion of the band, may be connected to the other end of the stretchable display 110. The portion of the band 121 that is connected to the other end of the stretchable display 110 may be provided with hook holes 125 that are spaced apart from each other at a predetermined interval along a length direction. The hook protrusion 123 may be inserted into the hook hole 125. For example, the user may insert the hook protrusion 123 into the hook hole 125 while changing a position of the hook hole 125 to fit for a size of his/her wrist or forearm.

The body sensor 130 may be used to sense a change in a human body. The body sensor 130 may output a sensing signal (depending on the change in a human body) to a driving controller 150 that is provided in the stretchable display 110.

The body sensor 130 may be installed on a rear (e.g., interior, body-facing side) of the stretchable display 110 that contacts a skin of, e.g., the body's wrist or forearm. The body sensor may include a moisture sensor 131 sensing sweat generated from the human body. The moisture sensor 131 may be provided as a humidity sensor that senses humidity due to sweat discharged from a skin of a user's wrist or a forearm. In an implementation, the body sensor 130 may include a temperature sensor 133 that senses body temperature. The temperature sensor 133 may sense heat diffused from the user's wrist or forearm to measure the body temperature. The moisture sensor 131 and the temperature sensor 133 may include a suitable humidity sensor and/or heat sensor.

In an implementation, both the moisture sensor 131 and the temperature sensor 133 of the body sensor 130 may be installed on the rear of the stretchable display 110. In this case, the moisture sensor 131 and the temperature sensor 133 may be alternately disposed along the length direction of the stretchable display 110.

In an implementation, the body sensor 130 according to the embodiment may include, e.g., the moisture sensor 131, the temperature sensor 133, a heart rate sensor, a blood pressure sensor, blood sugar sensor, and/or the like.

The modifier 140 may increase an area of the stretchable display 110 or may reduce the area thereof to an original area, based on the sensing signal generated from, e.g., the moisture sensor 131 and the temperature sensor 133 of the body sensor 130. For example, the modifier 140 may provide an external force to the stretchable display 110 based on the sensing signal generated from the moisture sensor 131 and the temperature sensor 133 of the body sensor 130, thereby increasing the length of the stretchable display 110 or restoring the length of the stretchable display 110 to an original state.

The modifier 140 may be installed on the rear (e.g., interior side) of the stretchable display 110. According to the exemplary embodiment, the modifier 140 may include electromagnets 141 that are installed inside the rear of the stretchable display 110. The electromagnets 141 may be spaced apart from each other at a predetermined interval along a stretched direction (e.g., the length direction) of the stretchable display 110

The electromagnets 141 may be disposed inside the rear of the stretchable display 110, may extend along or in a width direction of the stretchable display 110, and may be spaced apart from each other along the length direction of the stretchable display 110.

The electromagnet 141 may be applied with a current by the driving controller 150 to generate an electromagnetic force. The driving controller 150 may apply a current in a same direction to the electromagnet 141 based on the sensing signal from the moisture sensor 131 and the temperature sensor 133 of the body sensor 130. Therefore, the electromagnet 141 may form a same pole to generate a repulsive force and extend the length of the stretchable display 110 by the generated repulsive force. In an implementation, the driving controller 150 may reduce or interrupt a current applied to the electromagnet 141 based on the sensing signal generated from the moisture sensor 131 and the temperature sensor 133 of the body sensor 130. Thus, the electromagnet 141 may have the reduced repulsive force to thereby contract the stretchable display 110 to restore, e.g., reduce, the length of the stretchable display 110 back to an original state. In an implementation, the modifier 140 may include, e.g., the electromagnet 141 (that controls the length of the stretchable display 110), and/or various other suitable forms that may increase or reduce the length of the stretchable display 110.

As described above, the driving controller 150 may control a whole operation of the stretchable display 110 and may control the current applied to the electromagnet 141 of the modifier 140. The driving controller 150 may be applied with the sensing signal from the moisture sensor 131 and the temperature sensor 133 of the body sensor 130. The driving controller 150 may calculate the humidity of the user's wrist or forearm depending on the sensing signal of the moisture sensor 131 and may compare the humidity calculation value with a preset reference value. If it is determined that the humidity calculation value exceeds the reference value, the driving controller 150 may apply the current in the same direction to the electromagnet 141. If it is determined that the humidity calculation value is less than the reference value, the driving controller 150 may reduce or interrupt the current applied to the electromagnet 141. In an implementation, the driving controller 150 may calculate the temperature of the user's wrist or forearm depending on the sensing signal of the temperature sensor 133, and may compare the temperature calculation value with a preset reference value. If it is determined that the temperature calculation value exceeds the reference value, the driving controller 150 may apply the current in the same direction to the electromagnet 141. If it is determined that the temperature calculation value is less than the reference value, the driving controller 150 may reduce or interrupt the current applied to the electromagnet 141.

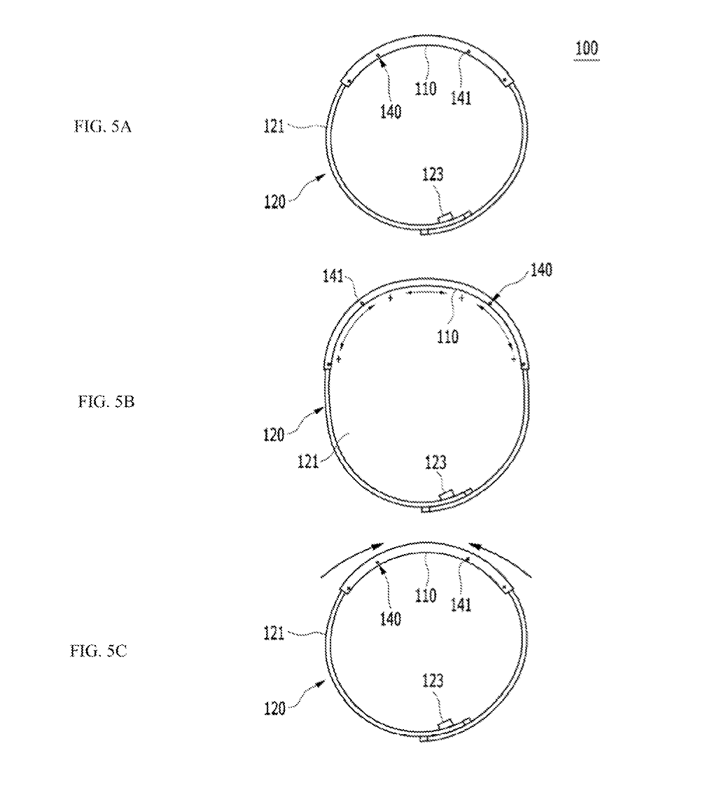

Hereinafter, referring to the drawings as described above and FIGS. 5A to 5C, an operation and an action effect of the wearable display device 100 according to the exemplary embodiment will be described.

First, referring to FIG. 5A, the stretchable display 110 may be put on the user's wrist or forearm using the band 121 of the wearing part 120. In this case, the user may control a wearing length of the band 121 to fit for a size of his/her wrist or forearm and wear the stretchable display 110. The wearing length of the band 121 may be controlled by inserting the hook protrusion 123 into the selected hook hole 125, e.g., while changing the position of the hook hole 125.

In this state, when the user wears the stretchable display 110 for a long period of time, or sweat is discharged from a skin of a user's wrist or forearm and body temperature is increased due to activities of an exercise, the moisture sensor 131 of the body sensor 130 may sense humidity due to sweat and may output the sensing signal to the driving controller 150. Next, the driving controller 150 may calculate the humidity of the user's wrist or forearm depending on the sensing signal of the moisture sensor 131, may compare the humidity calculation value with the preset value and, if it is determined that the humidity calculation value exceeds the reference value, may apply the current in the same direction to the electromagnet 141 of the modifier 140.

In an implementation, the temperature sensor 133 of the body sensor 130 may sense the body temperature and may output the sensing signal of the body temperature to the driving controller 150. Next, the driving controller 150 may calculate the temperature of the user's wrist or forearm depending on the sensing signal of the temperature sensor 133, may compare the temperature calculation value with the preset value and, if it is determined that the temperature calculation value exceeds the reference value, may apply the current in the same direction to the electromagnet 141 of the modifier 140.

Thus, the electromagnet 141 of the modifier 140 may be applied with the current in the same direction through the driving controller 150. Accordingly, as illustrated in FIG. 5B, as the same pole is formed in the electromagnet 141, the length of the stretchable display 110 may extend in response to the repulsive force.

Therefore, according to the exemplary embodiment, the length of the stretchable display 110 may be increased by the modifier 140 and thus a constant interval or space between the stretchable display 110 and the user's wrist or forearm may be formed. Therefore, according to the exemplary embodiment, air may flow in the interval between the stretchable display 110 and the user's wrist or forearm to help evaporate sweat and/or reduce body temperature.

Meanwhile, when the sweat discharged from the user's wrist or forearm is evaporated and the body temperature is reduced, the driving controller 150 may be provided with the sensing signal from the moisture sensor 131 and the temperature sensor 133 and, if it is determined that the humidity calculation value and the temperature calculation value are less than the reference value, may reduce or interrupt the current applied to the electromagnet 141. Therefore, the electromagnet 141 of the modifier 140 may reduce the repulsive force and, as illustrated in FIG. 5C, may restore the length of the stretchable display 110 back to an original state.

In the wearable display device 100 according to the exemplary embodiment as described above, the body sensor 130 may sense the change in a human body and may automatically control the length of the stretchable display 110 using the modifier 140, depending on the change in a human body.

Therefore, in the wearable display device 100 according to the exemplary embodiment, it is possible to reduce and/or eliminate the inconvenience of the uncomfortable wearing feeling due to the long term wearing of the stretchable display 110, during activities of exercise or the like.

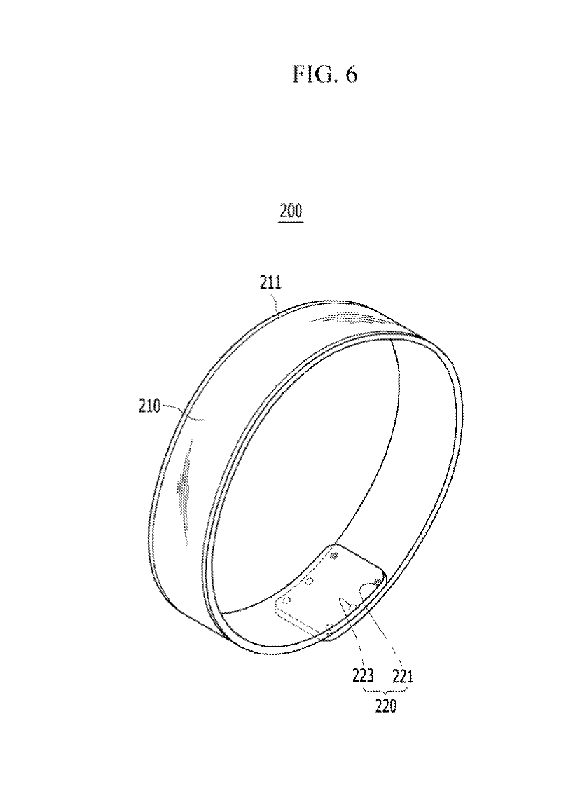

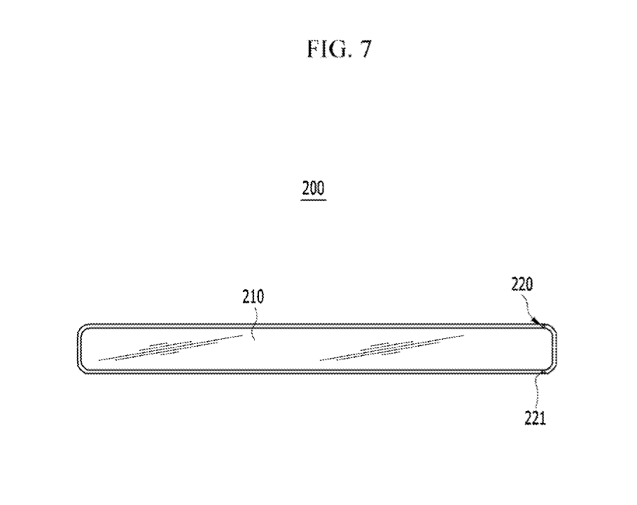

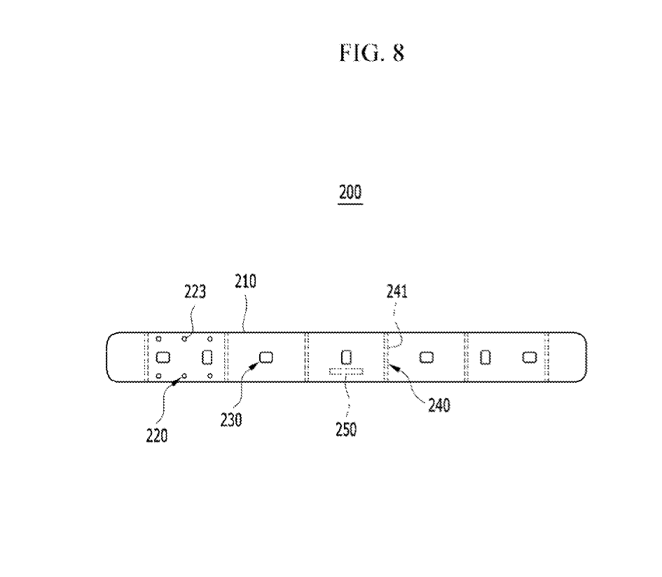

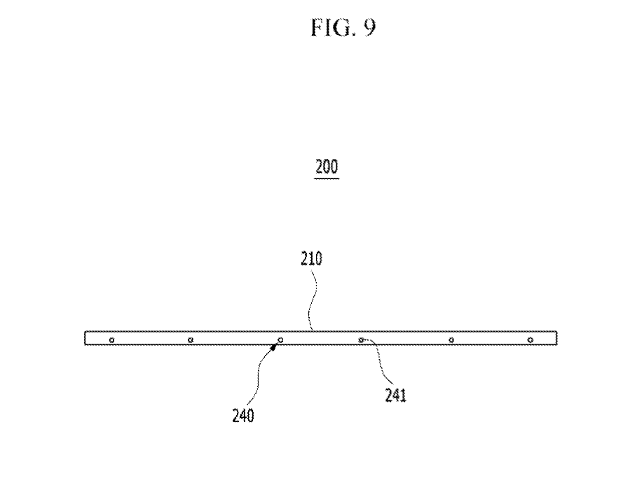

FIG. 6 illustrates a perspective view of a wearable display device according to another exemplary embodiment. FIG. 7 illustrates a front configuration diagram of the wearable display device according to other exemplary embodiment. FIG. 8 illustrates a rear configuration diagram of the wearable display device according to other exemplary embodiment. FIG. 9 illustrates a side configuration diagram of the wearable display device according to other exemplary embodiment.

Referring to FIGS. 6 to 9, a wearable display device according to other exemplary embodiment may include a stretchable display 210, a wearing part 220, a body sensor 230, a modifier 240, and a driving controller 250.

A configuration of the stretchable display 210, the body sensor 230, and the modifier 240 may be the same as the exemplary embodiment described above, and therefore a repeated detailed description thereof may be omitted.

In an implementation, the wearing part 220 may be used to put or attach the stretchable display 210 itself onto the user's wrist or forearm. For example, the wearing part 220 may be provided in the stretchable display 210 itself. The wearing part 220 may be provided at edge portions 211, e.g., both edge portions 211, along the length direction of the stretchable display 210. For example, the wearing part 220 may be provided at upper and lower edge portions, as illustrated in FIGS. 7 and 8. The wearing part 220 may include, e.g., magnetic bodies 221 and a metal body 223.

The magnetic bodies 221 may be, e.g., a permanent magnet having magnetism and/or may be an electromagnet which is applied with a current to generate an electromagnetic force. The magnetic bodies 221 may be installed at, e.g., both, edge portions 211 of one end of the stretchable display 210. The magnetic bodies 221 may be penetratedly inserted and fixed into both edge portions of a front of the stretchable display 210 and/or may be attached to surfaces of the both edge portions 211.

The metal bodies 223 may be attached, attracted, or coupled to the magnetic bodies 221 by a magnetic force of the magnetic bodies 221, and may be installed at, e.g., both, edge portions of the other edge portion 211 of the stretchable display 210 to be spaced apart at a predetermined interval. The metal bodies 223 may be penetratedly inserted and fixed into both edge portions of a rear of the stretchable display 210 and/or may be attached to surfaces of the both edge portions 211.

Therefore, the user may attach or couple the metal body 223 to the magnetic body 221 while changing the position of the metal body 223 to fit for his/her wrist or forearm size to put the stretchable display 210 on his/her wrist or forearm.

Meanwhile, when the stretchable display 210 according to the present embodiment is put on the body's wrist or forearm by way of the wearing part 220, one end or portion of the display area may overlap another end or portion of the display area by a predetermined section, depending on the size of the wrist or forearm. In this case, the overlapped portions of the stretchable display 210 may be fastened with each other through the magnetic body 221 and the metal body 223 of the wearing part 220.

The stretchable display 210 may display an image in the remaining area, e.g., other than the overlapped portion. For example, the image may not be displayed at the overlapped portion, and the image may be continuously displayed in the remaining area. The display driving of the stretchable display 210 may be implemented by the driving controller 250. Suitable technologies, e.g., a technology of recognizing the overlapped point of the stretchable display 210 by a change in a charging rate of the touch panel electrode, may be applied to the driving controller 250.

In an implementation, the driving controller 250 may apply the current in the same direction to the electromagnet 241 of the modifier 240 based on the sensing signal of the body sensor 230. A configuration and an operation of the modifier 240 may be the same as the foregoing exemplary embodiments and therefore a repeated detailed description thereof may be omitted. In an implementation, the driving controller 250 may recognize the overlapped point of the stretchable display 210 so as not to apply the current to the electromagnet 241 that is positioned at the overlapped portion of the stretchable display 210. The reason is that if a current were to be applied even to the electromagnet 241 at the overlapped portion of the stretchable display 210, the overlapped portion could extend in response to the force applied by the electromagnet 241. If the overlapped portion of the stretchable display 210 were to extend, the magnetic body 221 of the wearing part 220 could be separated from the metal body 223, such that the stretchable display 210 could be separated from the wrist or the forearm.

Other configurations and action effects of the wearable display device 200 according to the present embodiment may be the same as the foregoing exemplary embodiments, and therefore a repeated detailed description thereof may be omitted.

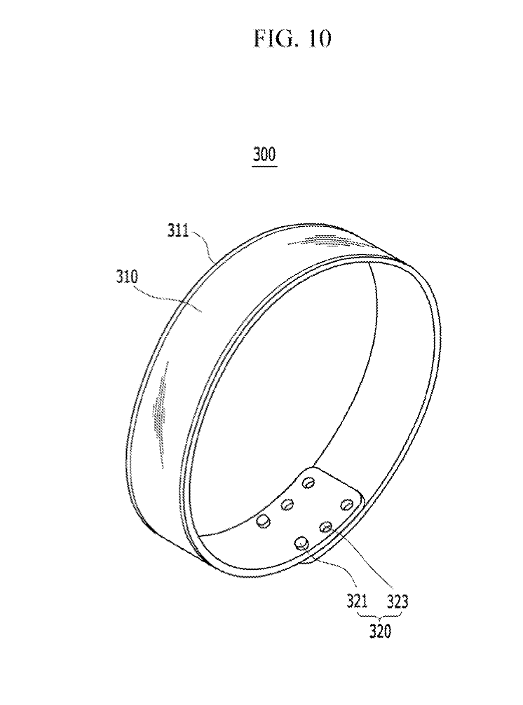

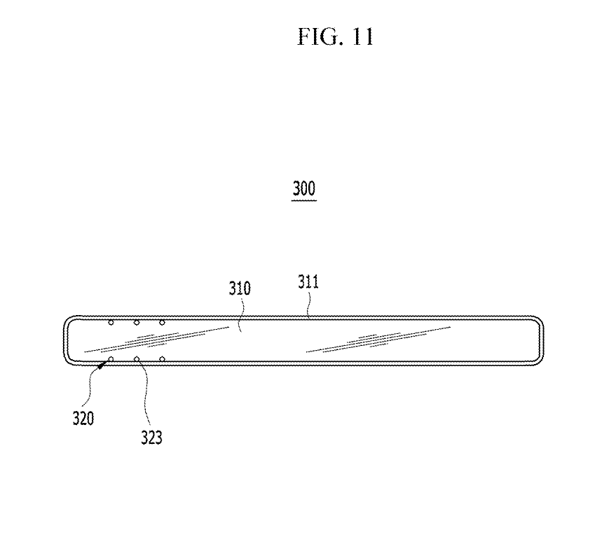

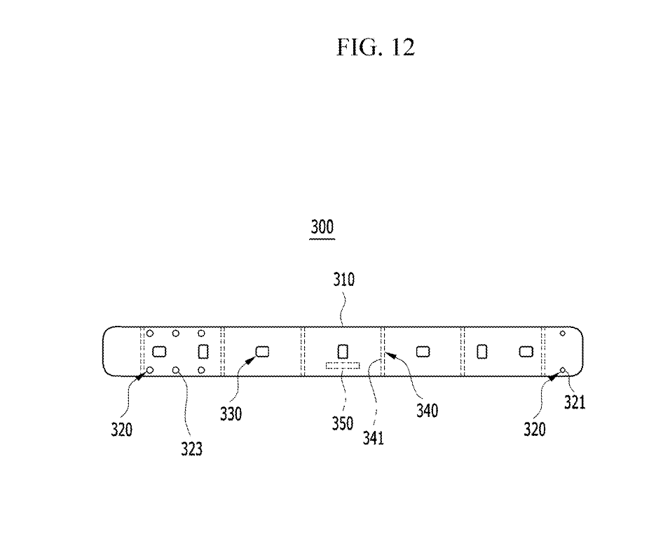

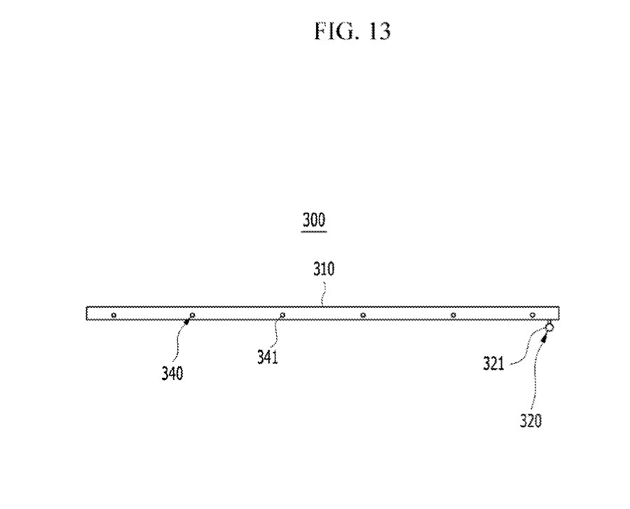

FIG. 10 illustrates a perspective view of a wearable display device according to still another exemplary embodiment. FIG. 11 illustrates a front configuration diagram of the wearable display device of FIG. 10. FIG. 12 illustrates a rear configuration diagram of the wearable display device of FIG. 10. FIG. 13 illustrates a side configuration diagram of the wearable display device of FIG. 10.

Referring to FIGS. 10 to 13, a wearable display device 300 according to still another exemplary embodiment may include, e.g., a stretchable display 310, a wearing part 320, a body sensor 330, a modifier 340, and a driving controller 350.

A configuration of the stretchable display 310, body sensor 330, and modifier 340 may be the same as the previous embodiments, and therefore a repeated detailed description thereof may be omitted.

According to the present embodiment, the wearing part 320 may be used to put or attach the stretchable display 310 itself onto, e.g., the user's wrist or forearm. The wearing part 320 may be provided in the stretchable display 310 itself. The wearing part 320 may be provided at, e.g., both, edge portions 311 along the length direction of the stretchable display 310. For example, the wearing part 320 may be provided at upper and lower edge portions as illustrated in FIGS. 11 and 12. The wearing part 320 may include hook protrusions 321 and hook holes 323.

The hook protrusions 321 may each be installed at both edge portions 311 of one end of the stretchable display 310. The hook protrusion 321 may protrude at the, e.g., both, edge portions 311 on a rear of the stretchable display 310 to be integrally formed with the stretchable display 310, or may be fixedly installed to both edge portions 311 as a separate component.

The hook protrusions 321 may be inserted into the hook holes 323 that are formed at, e.g., both, edge portions 311 of the other end of the stretchable display 310 to be spaced apart from each other at a predetermined interval.

Therefore, the user may insert the hook protrusion 321 into the hook hole 323 while changing the position or selection of the hook hole 323 to fit for his/her wrist or forearm to put the stretchable display 310 on his/her wrist or forearm.

Meanwhile, when the stretchable display 310 according to the present embodiment is put on the body's wrist or forearm through the wearing part 320, the display area may overlap itself by a predetermined section, depending on the size of the wrist or forearm. In this case, the overlapped portion of the stretchable display 310 may be fastened with each other through the hook protrusion 321 and the hook hole 323 of the wearing part 320.

The stretchable display 310 may display an image in the rest or remaining area, other than the overlapped portion. For example, the image may not be displayed at the overlapped portion and the image may be continuously displayed in the remaining area. The display driving of the stretchable display 310 may be implemented by the driving controller 350. Suitable technologies, e.g., a technology of recognizing the overlapped point of the stretchable display 310 by a change in a charging rate of the touch panel electrode, may be applied to the driving controller 350.

In an implementation, the driving controller 350 may apply the current in the same direction to the electromagnet 341 of the modifier 340 based on the sensing signal of the body sensor 330. A configuration and an operation of the modifier 340 may be the same as the previous embodiments, and therefore a repeated detailed description thereof may be omitted. In an implementation, the driving controller 350 may recognize the overlapped point of the stretchable display 310 so as not to apply the current to the electromagnet 341 that is positioned at the overlapped portion of the stretchable display 310. The reason is that if a current were to be applied even to the electromagnet 341 at the overlapped portion of the stretchable display 310, the overlapped portion could be extended due to the force of the electromagnet 341. If the overlapped portion of the stretchable display 310 were to extend, the hook protrusion 321 of the wearing part 320 could be separated from the hook hole 323, such that the stretchable display 310 could be separated from the wrist or the forearm.

The remaining configurations and action effects of the wearable display device 300 according to the present embodiment may be the same as the previous embodiments, and therefore a repeated detailed description thereof may be omitted.

By way of summation and review, the wearable device may be put on to fit for a size of a wrist or a forearm using a band, and the user may feel inconvenience due to sweat, body temperature, external temperature, etc., when the user wears the wearable device for a long period of time.

The embodiments may provide a wearable display device mounted on a user's wrist or forearm to display an image.

The embodiments may provide a wearable display device having advantages of automatically controlling a size of a display in response to a change in a human body.

As described above, according to an embodiment, the wearable display device may sense the change in a human body using the sensor and automatically control the length of the stretchable display in response to the change in a human body using the modifier, thereby reducing and/or eliminating inconvenience of the wearing feeling due to the long term wearing of the stretchable display or the activities such as exercise.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

### DESCRIPTION OF SYMBOLS

100, 200, 300 Wearable display device

110, 210, 310 Stretchable display

120, 220, 320 Wearing part

121 Band

123, 321 Hook protrusion

125, 323 Hook hole

130, 230, 330 Body sensor

131 Moisture sensor

133 Temperature sensor

140, 240, 340 Modifier

141, 241, 341 Electromagnet

150, 250, 350 Driving controller

211, 311 Both edge portions

221 Magnetic body

223 Metal body

What is claimed is:

1. A wearable display device, comprising:
a stretchable display wearably providable on a human body to display an image;
a body sensor on a rear of the stretchable display to contact the human body; and
a modifier in the stretchable display, the modifier increasing an area of the stretchable display based on a sensing signal generated from the body sensor.

2. The wearable display device as claimed in claim 1, wherein:
the stretchable display is stretchable and contractible in a length direction thereof in response to an external force, and
the modifier provides the external force to the stretchable display to increase a length of the stretchable display or restore the length of the stretchable display to an original state.

3. The wearable display device as claimed in claim 1, wherein the body sensor includes a moisture sensor to sense sweat generated from the human body.

4. The wearable display device as claimed in claim 1, wherein the body sensor includes a temperature sensor to sense body temperature.

5. The wearable display device as claimed in claim 1, wherein the body sensor includes:
a moisture sensor to sense sweat generated from the human body, and
a temperature sensor to sense body temperature.

6. The wearable display device as claimed in claim 5, wherein the moisture sensor and the temperature sensor are alternately disposed along a length direction of the stretchable display.

7. The wearable display device as claimed in claim 1, wherein the modifier includes electromagnets inside a rear of the stretchable display, the electromagnets being spaced apart from each other at a predetermined interval along a length direction of the stretchable display.

8. The wearable display device as claimed in claim 7, wherein:
the body sensor senses a change in the human body and outputs the sensing signal to a driving controller of the stretchable display, and
the driving controller applies a current in a same direction to the electromagnet based on the sensing signal.

9. The wearable display device as claimed in claim 8, wherein the electromagnet forms a same pole to generate a repulsive force and extend a length of the stretchable display by the generated repulsive force.

10. The wearable display device as claimed in claim 1, wherein the stretchable display includes an organic light emitting diode.

11. The wearable display device as claimed in claim 1, further comprising wearing parts provided at sides of the stretchable display to put the stretchable display on the human body.

12. The wearable display device as claimed in claim 11, wherein the wearing part includes a band that is connected to sides of the stretchable display.

13. The wearable display device as claimed in claim 12, wherein the band includes:
one portion that is connected to one end of the stretchable display and that has a rear provided with a hook protrusion, and
another portion that is connected to another end of the stretchable display and that is provided with hook holes that are spaced apart from each other along a length direction of the band.

14. The wearable display device as claimed in claim 11, wherein the wearing parts are provided at edge portions along a length direction of the stretchable display.

15. The wearable display device as claimed in claim 14, wherein the wearing part includes:
magnetic bodies installed at the edge portion of one end of the stretchable display; and
metal bodies installed at the edge portion of another end of the stretchable display to be spaced apart from each other at a predetermined interval.

16. The wearable display device as claimed in claim 14, wherein the wearing part includes:
hook protrusions installed at the edge portions of one end of the stretchable display; and
hook holes installed at the edge portions of another end of the stretchable display to be spaced apart from each other at a predetermined interval.

17. The wearable display device as claimed in claim 11, wherein:
opposing ends of the wearing parts overlap at an overlapped portion,
the stretchable display displays an image in a remaining area other than an overlapped portion, and
the driving controller does not apply a current to the electromagnet that is positioned at the overlapped portion of the wearing parts.
